(19) United States
(12) Patent Application Publication
(10) Pub. No.: US 2008/0288060 A1
(43) Pub. Date: Nov. 20, 2008

(54) Treating Valvular Insufficiency

(75) Inventors: David Martin Kaye, Victoria (AU); John Melmouth Power, Victoria (AU); Clifton A. Alferness, Port Orchard, WA (US); Adam Lucas Bilney, Victoria (AU); Mark L. Mathis, Fremont, CA (US)

Correspondence Address: MERCHANT & GOULD PC, P.O. BOX 2903, MINNEAPOLIS, MN, 55402-0903, US

(73) Assignee: Baker Medical Research Institute, Prahran, Victoria (AU)

(21) Appl. No.: 11/631,832

(22) PCT Filed: Jul. 6, 2005
(86) PCT No.: PCT/AU2005/000992

§ 371 Date: Mar. 12, 2008

Related U.S. Application Data
(60) Provisional application No. 60/585,784, filed on Jul. 6, 2004.

(51) Int. Cl. A61F 2/24

(52) U.S. Cl. 623/2.36

(57) ABSTRACT

In a method of treating valvular insufficiency in a patient, a plurality of filaments (2) are used to engage tissue at spaced apart locations of an annulus (1) of the valve being treated. The engaged filaments (2) are drawn inward so as to draw the engaged tissue around the valve annulus (1) inward. The filaments (2) are then secured with the engaged tissue in the drawn-in configuration. Inward drawing of the engaged tissue improves valve function by reducing the valve annulus (1). Alternatively or additionally, anchor means may be used to secure the filaments to a region of robust tissue, thus drawing the engaged tissue toward the anchor means to further improve valve function.

### FIELD OF THE INVENTION

The present invention relates to a method and apparatus for treating valvular insufficiency. In particular, the invention relates to a device and method for treating valvular insufficiency occurring in valves of the heart such as the tricuspid valve, and a delivery apparatus for the same.

### BACKGROUND TO THE INVENTION

The body's circulation is facilitated by the heart, the cardiac pump which ensures that fresh blood is supplied throughout the body delivering nutrients to organs and transporting waste products to the body's filtration systems. The heart, simplified and illustrated in cross section in FIG. 1, is a complex organ operating two pumping systems. One pumping system includes the left ventricle (LV) and left atrium (LA) and services the systemic circulation in which oxygenated blood is supplied to the body's organs. Deoxygenated blood is then returned to the right heart. The other pumping system includes the right ventricle (RV) and right atrium (RA) and services the pulmonary circulation, pumping deoxygenated blood from the heart to the lungs where it is re-oxygenated and then returned to the left heart for re-circulation to the body's organs.

Valves in the heart and throughout the body ensure that blood flows constantly in one direction. These include the mitral valve and the tricuspid valve, which separate the atria and ventricles of the left and right hearts respectively. The circulation is dependent on these valves to ensure that the blood is pumped continuously and efficiently through the heart and delivered to the rest of the body.

The tricuspid valve is a complex structure comprising leaflet tissue, chordae tendinae, papillary muscles and a supporting annulus. The tricuspid valve leaflets are a continuous veil of leaflet tissue that attach to the annulus. Three major leaflets are identified, anterior, septal and posterior. The tricuspid valve annulus performs multiple functions including maintenance of valvular shape and dimensions.

In some cases, valves in the circulatory system such as the tricuspid and mitral heart valves are deficient or fail. The causes of partial or total heart valve failure include congenital/structural defects, disease and infection. However, the most common cause of valve failure is dilation of the valve annulus. This occurs as part of the generalized cardiac structural dilatation allied to cardiomyopathy and heart failure. The consequences of heart valve failure can vary depending on the seriousness of the failure, but in most cases the heart's efficiency and the efficiency of the circulatory system is seriously affected and complications often result.

Failure or insufficiency of the heart valves frequently results in mitral/tricuspid valve regurgitation. In the case of the mitral valve, regurgitation results in back pressure in the lungs, whereas tricuspid valve regurgitation can result in high back pressures in the venous circulation. Clearly, this is undesirable for the health of the heart, as well as for the lungs and other organs of the body. Mitral and tricuspid valve failure can lead to ineffective and/or inefficient cardiac pumping, ventricular and atrial enlargement, pulmonary and/or circulatory hypertension, heart failure and in some cases, death.

Methods exist for repairing and replacing cardiac valves and other valves of the body and treatments for mitral valve regurgitation in particular are available. One form of treatment involves replacement of the entire valve. In other cases, the mitral or tricuspid valve annulus may be repaired by placing a biocompatible annuloplasty ring inside the annulus and suturing the ring to the fibrous tissue of the annulus. The annuloplasty ring constricts the annulus, enabling the mitral or tricuspid valve leaflets to seal during each pumping cycle and reduce or prevent backflow.

Mitral valve replacement and implantation of the annuloplasty ring both require open heart surgery and are therefore major operations. The patient must be placed under general anesthetic and undergo cardiopulmonary bypass.

Concomitant with the seriousness of such procedures are an increase in morbidity and mortality risk, and a slow and painful period of rehabilitation which follows. Post-operative complications are also common and these include infection, thromboembolism, loss of ventricular function and a need for anticoagulation medication.

The location of the tricuspid valve in the right heart complicates treatment because it is less easily accessible than the mitral valve, and it has a more complex triple-leaved structure. The mitral valve is accessible via the coronary sinus/great cardiac vein (CS/GCV) which has a close anatomical relationship with the lateral border of the posterior annulus. The small cardiac vein has a similar relationship with the tricuspid annulus. However, unlike the CS/GCV, this vessel is small, variable in size and absent in approximately 50% of cases. Therefore, reasonable vascular access to the tricuspid annulus is limited to a right atrial approach.

In the past, implications of tricuspid valve regurgitation have not been well understood, and this has only become a topic of interest in recent times. Because of this, the treatment options available for patients experiencing tricuspid valve regurgitation are limited. Currently available forms of treatment for patients experiencing mitral and tricuspid valve insufficiency are high risk, expensive and prone to complications.

### SUMMARY OF THE INVENTION

Briefly, a first aspect of the present invention provides a method of treating valvular insufficiency in a patient. A plurality of filaments is used to engage tissue at spaced apart locations of an annulus of the valve being treated. The engaged filaments are drawn inward so as to draw the engaged tissue inward. The filaments are then secured with the engaged tissue in the drawn-in configuration. Inward drawing of the engaged tissue improves valve function by reducing the valve annulus.

A second aspect of the present invention provides a valve constriction device for treating valvular insufficiency. The valve constriction device has a plurality of filaments, each having an engaging portion for engaging annular tissue of the valve. A collar is also provided, facilitating a drawn-in configuration of engaged annular tissue by relative movement between the filaments and the collar. Valve function is improved when the engaged filaments are in the drawn-in configuration.

A third aspect of the present invention provides delivery apparatus for delivering a valve annulus constriction device. The apparatus includes a catheter configured to deliver a plurality of filaments percutaneously to an annulus of the valve being constricted. The catheter is also configured to deliver a collar to be applied over a length of the filaments. Relative movement between the catheter and a filament facilitates positioning of the filament prior to engagement with the valve annulus tissue.

A fourth aspect of the present invention provides a method of treating valvular insufficiency in a patient in which a plurality of filaments is used to engage tissue at spaced apart locations of an annulus of the valve being treated. The plurality of filaments are then anchored to a region of robust tissue near the valve being treated so as to draw the engaged tissue toward the anchor region thus improving closure of the valve being treated.

A fifth aspect of the present invention provides a valve constriction device for treating valvular insufficiency which includes a plurality of filaments. Each filament has an engaging portion for engaging annular tissue of the valve. Anchor means is also provided for anchoring the engaged filaments to a region of robust tissue to facilitate drawing of the engaged tissue toward the anchor means to improve leaflet closure.

### BRIEF DESCRIPTION OF THE DRAWINGS

FIG. 1 illustrates a simplified cross section of the heart.

FIG. 2 shows steps in a method of treating valvular insufficiency according to an embodiment of the invention.

FIGS. 3A and 3B illustrate a valve constriction device according to an embodiment of the invention with the engaged valve annulus tissue in FIG. 3B in a drawn-in configuration.

FIG. 4 illustrates a delivery apparatus for delivering the valve constriction device of FIGS. 3A and 3B.

FIGS. 5A and 5B illustrate positioning and engagement of engaging portions with the valve annulus tissue, with FIG. 5B illustrating in particular, splay of one the sheathed filaments.

FIGS. 6A and 6B illustrate a constriction device and a secondary support structure according to embodiments of the invention.

FIG. 7 illustrates the valve constriction device of FIGS. 3A and 3B including a locking member.

FIGS. 8A and 8B show cross sectional views of one form of locking member in its unlocked and locked conditions respectively.

FIG. 9 illustrates an embodiment of the invention with the constriction device anchored in the superior vena cava.

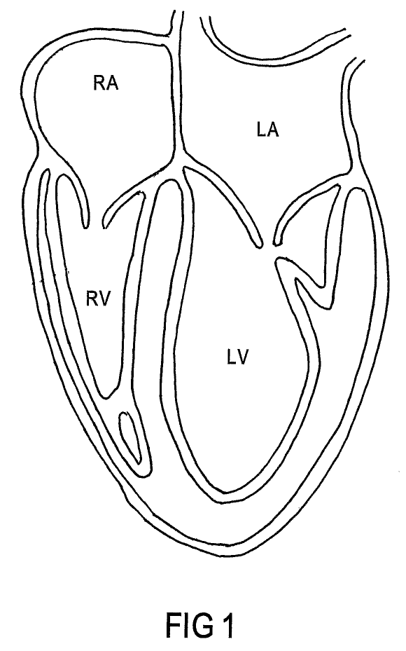

### DETAILED DESCRIPTION OF THE INVENTION

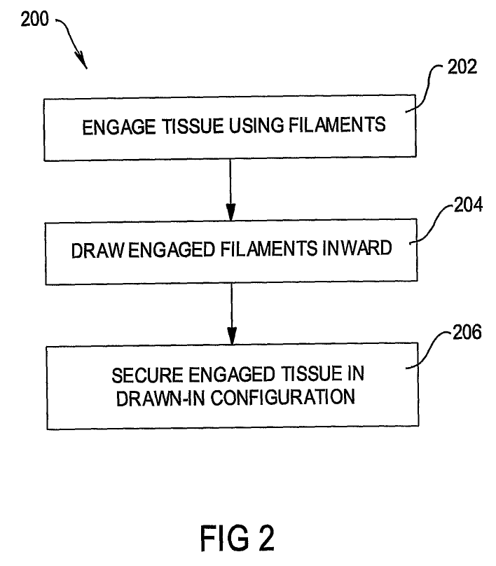

Referring firstly to FIG. 2, there is shown a method, generally referred to at 200, for treating valvular insufficiency. In a first step 202, a plurality of filaments is used to engage tissue at spaced apart locations of an annulus of the valve being treated. In a second step 204, the engaged filaments are drawn inward so as to draw the engaged tissue around the valve annulus inward. The engaged filaments are then secured with the engaged tissue in the drawn-in configuration in a third step 206 thereby reducing the effective diameter of the annulus of the valve.

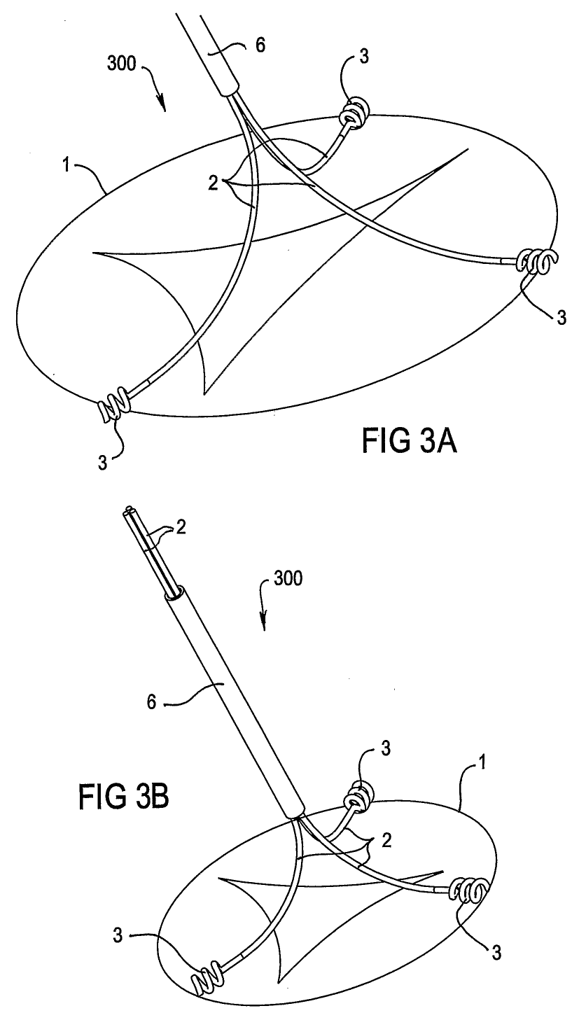

Referring now to FIGS. 3A and 3B, there is shown a valve constriction device 300 for treating valvular insufficiency according to an embodiment of the invention. A plurality of filaments 2 each have an engaging portion 3 for engaging annular tissue 1 of the valve being treated. A collar 6 facilitates a drawn in configuration of filaments 2 by relative movement between the filaments and the collar. The valve annulus is substantially constricted, as shown in FIG. 3B when the engaged filaments are in the drawn in configuration thus restoring or improving valve closure.

The embodiments illustrated in the accompanying drawings demonstrate use of the invention in its various embodiments to treat insufficiency of the tricuspid valve. It is to be understood, however, that the method is also suitable for other valves of the heart and for treating insufficiency of a range of other valves around the body. Other valves for which the invention may be suitable may include but are not limited to the valves of the esophagus, urinary tract and intestinal tract.

In order to minimize the invasiveness of the procedure, it is desirable that the method is performed percutaneously. That is, using a catheter or other such lumen which is sufficiently flexible to enter the patient's circulation through the skin and into the jugular vein or other blood vessel, and to be directed to the valve being treated. For treatment of the tricuspid valve, it is preferred that the device is transported to the tricuspid valve annulus 1 through the right atrium.

It has been found that for treatment of tricuspid valve insufficiency, the method is effective when three filaments are used. However, it is to be understood that use of other quantities of filaments may be used to achieve the desired outcome of improved or restored valve function. The number of filaments used may depend, for example on the size of the patient and/or the size and geometry of the valve being treated.

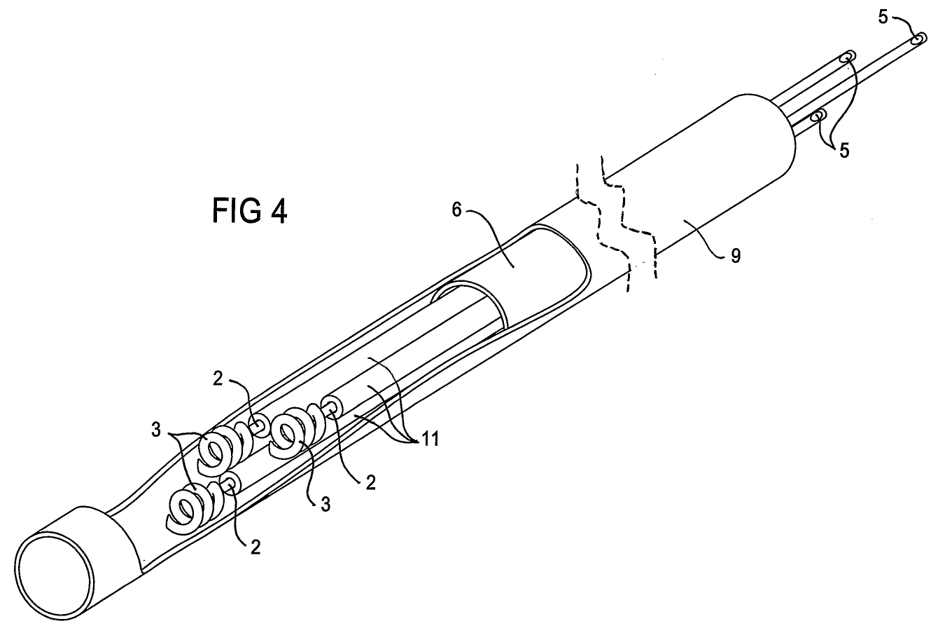

In FIG. 4 there is shown a delivery apparatus in the form of catheter 9 containing a valve constriction device according to an embodiment of the invention. Filaments 2 are positioned within the catheter 9 so that the engaging portions 3 are in a staggered formation. This reduces the likelihood of the engaging portions (shown in the form of helical tips) 3 becoming tangled or caught within catheter 9 during delivery of the constriction device through the vascular system to the valve. Also, the staggered arrangement facilitates miniaturization of the device and delivery apparatus, by reducing the overall diameter required to contain the filaments and engaging portions during delivery.

Whilst the engaging portions 3 are illustrated throughout the embodiments illustrated as including a helical tip, it is to be understood that the engaging portion of each of the filaments may be provided in any other suitable form whilst remaining within the scope of the claims appended hereto. Such alternative forms may include hooks, barbs, spikes or other suitable engaging means. Preferably, the tip of each engaging portion is pointed or otherwise configured for ease of engagement with the fibrous tissue of the valve annulus. In some embodiments, depending on the structure of the engaging portions, staggering the engaging portions within the guide catheter 9 may be less advantageous or even unnecessary.

The delivery apparatus may also include a percutaneously operable tool such as a torque tool for engaging the engaging portions with the annular tissue. Such a tool may be releasably connectable with each of the filaments. In one embodiment, filaments 2 each have a finite length with the engaging portion 3 at an annular end and a connecting region 5 at an opposing end. Engaging portion 3 is configured to engage tissue of the valve annulus. Meanwhile, connecting regions 5 are configured to releasably connect a torque and/or other tool used to position the filament and/or facilitate engagement of the associated engaging portion with the annular tissue. One such embodiment illustrated in FIG. 4 shows connecting portion 5 in the form of an eyelet adapted to connect a suitable tool to control positioning and engagement of the engaging portion with the valve annulus.

As briefly mentioned, for treatment of the tricuspid valve, it is preferred that catheter 9 enters the body through the jugular vein and snakes its way into the superior vena cava and right atrium to access the tricuspid valve annulus for repair. Surgeons or physicians performing the procedure may use any suitable imaging technique to view and assess the region of the valve annulus, to position the delivery apparatus and to deploy the constriction device in engagement the valve annulus tissue. X-ray fluoroscopy is one imaging technique which may be used to assist in accurately positioning the device. Alternatively or additionally, the physician may use haptic feedback and/or ancillary devices delivered to the region via catheter 9 or an additional lumen to position the delivery apparatus and constriction device and engage the engaging portions 3 of filaments 2 with the valve annulus tissue 1.

One or more of the filaments may also include an identifier visible from outside the body to enable the physician to correctly select a filament during deployment of the device. The identifiers may be in the form of a tag attached at the physician's end of each of the filaments, with a letter or number or other identifier on the tag to indicate which of the engaging portions located near the annulus corresponds to the filament being identified by the physician. Alternatively, the filaments may be color coded. In some embodiments, it may also be desirable for one or more of the engaging portions to include a radio-opaque marker. It is to be understood that a combination of 2 or more of these identification approaches, or other approaches as would be known to the person skilled in this area, may be adopted.

Catheter 9 is configured to deliver a plurality of filaments 2 percutaneously to the valve annulus 1 and preferably, to deliver a collar 6 over the filaments also. Preferably, each of the filaments 2 is individually covered in a sheath 11. Such sheath imparts strength to the filament 2 and provides a degree of rigidity which aids in positioning the engaging portions 3 prior to engagement with the annulus tissue.

Preferably, sheathed filaments 2 within catheter 9 are pre-formed in such a way that when catheter 9 is retracted by a small amount to reveal a length of sheathed filament 16 the sheathed filaments splay, curving away from the tip of the catheter. This may be achieved using any suitable technique, such as shape memory coding. Additionally or alternatively, filaments 2 may be formed with a taper, decreasing in cross-sectional area toward the engaging portion. This too may impart strength to the filaments without significantly affecting miniaturization of the device.

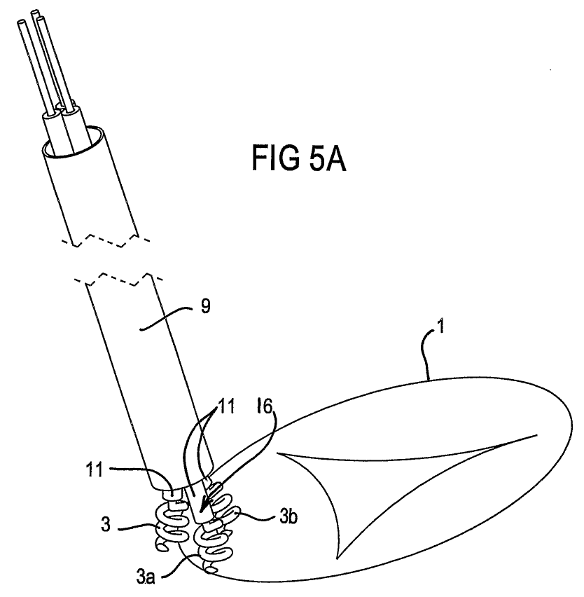

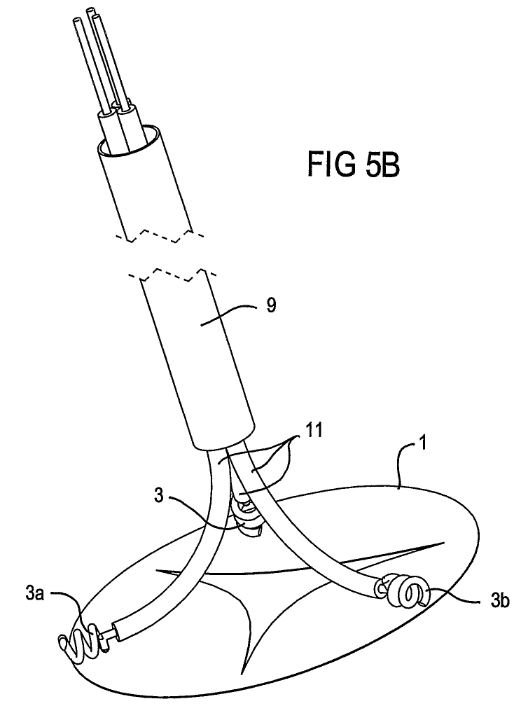

Positioning of the filaments prior to engagement with the valve annulus tissue may be controlled by relative movement between the catheter 9 and one or more of the sheathed filaments. Preferably this is achieved by slightly retracting catheter 9 relative to the filament being positioned as illustrated in FIGS. 5A and 5B.

FIG. 5A shows retraction of catheter 9 relative to one of the filaments revealing an engaging portion 3a which is subsequently made to engage with a portion of the valve annulus 1. FIG. 5B illustrates catheter 9 positioned in the valve annulus region and slightly retracted with respect to the filaments connected to engaging portions 3a and 3b. Engaging portion 3a already engaged with annular tissue (see FIG. 5A) remains in position with the engaged tip splaying away from catheter 9 whilst engaging portion 3b also splays away from the slightly retracted catheter so that it can be positioned and made to engage with the annular tissue. This process of retraction of the catheter and splay of the filament may be repeated for the remaining filament. In some instances it may be desirable to retract the catheter 9 relative to 2 or more of the filaments simultaneously to position the device's engaging portions. Splay of the filament may include splay of the sheath covering a length of the filament.

The delivery apparatus may also include a centering member such as a guide wire (not shown) with a tip locatable on a distal side of the valve being constricted to facilitate centralization of the constriction device relative to the valve annulus. When treating the tricuspid valve, the centering member would be temporarily anchored in the right ventricle during deployment of the constriction device.

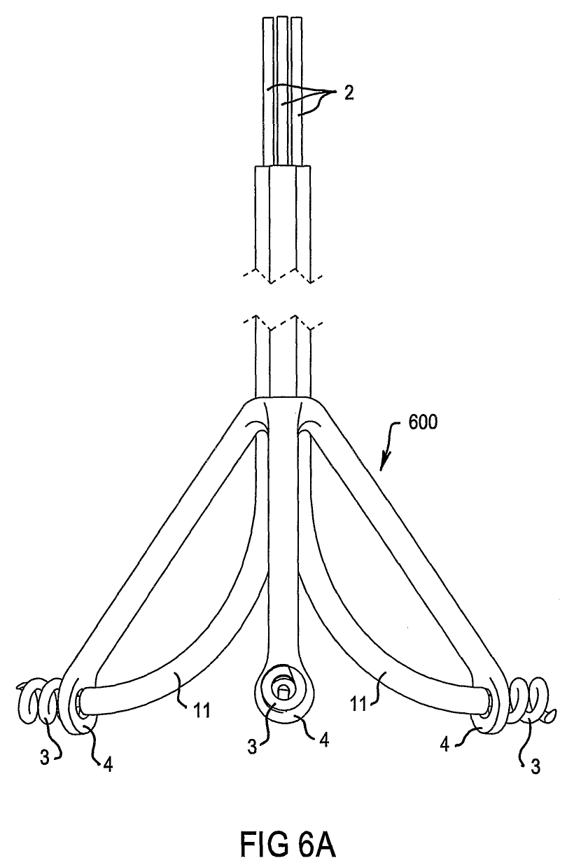

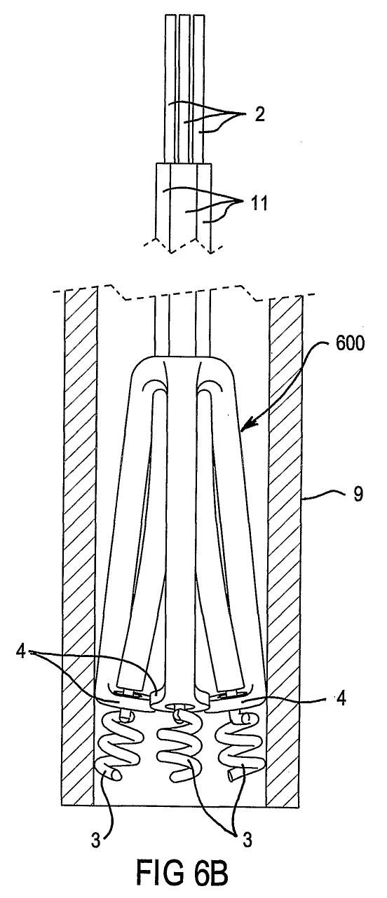

During engagement of one or more engaging portions with the annular tissue, torsional forces may develop, causing unengaged filaments to twist and making it difficult for the engaging portions to be positioned accurately. This torsional effect may be reduced by using a secondary support structure to support the unengaged filaments, thereby limiting twisting thereof. One such secondary support structure generally referred to as 600 is illustrated in FIGS. 6A and 6B. In FIG. 6A, the secondary support structure, having three arms each with an eyelet 4 at its end, is shown fitted to three sheathed filaments 2,11. It is desirable for the secondary support structure 600 to be formed from a material which resists torsional forces so that the arms do not twist during engagement of the individual engaging portions with the annular tissue. The support structure may be left in situ supporting the filaments after implantation of the constriction device. Alternatively the support structure may be used as a positioning aid which is removed after the filaments have been engaged with the annular tissue.

In one embodiment, one or more of the filaments may also be configured to conduct an electrical signal. Accordingly, when an engaging portion associated with such a filament is engaged with tissue of the heart, it will conduct electrical impulses propagating through that region of the heart. This signal can then be used as input to an analysis device to determine if the engaging portion of the filament is correctly positioned, based on the well-defined electrical characteristics of the heart. This feature is particularly useful for accurately positioning the engaging portion of a filament in the septal region of the annulus. Other techniques for accurately positioning the engaging portion 3 of each of the filaments may also be used. One such technique is to include a radio opaque marker with the engaging portions 3.

When each of the engaging portions 3 have been positioned at spaced apart locations of the valve annulus, a collar 6 having a relatively small diameter is delivered over a length of the filaments 2. In some embodiments it may be desirable to leave sheaths 11 in situ to impart further rigidity to the filaments and reduce the bending forces which would otherwise be borne by the filaments. In other embodiments it may be desirable to remove sheaths 11 with catheter 9, and apply collar 6 over the filaments as illustrated in FIGS. 3A and 3B.

Relative movement between the collar and the filaments draws the engaged tissue inward. This relative movement may involve moving the collar toward the annulus, or retracting the filaments through the collar, or by a combination of these. As the collar 6 approaches the annulus 1, the engaged tissue, is radially drawn-in to reduce the valve annulus. When the engaged tissue has been sufficiently drawn-in to restore or improve valve function, the filaments are retained in the drawn-in configuration, thereby treating the insufficiency problem. During deployment of the device, some physicians may find it useful, when the collar 6 is close to the annulus 1, to pull back on the filaments slightly. This has the combined effect of lifting annulus 1 and drawing the engaged annular tissue further inward, aiding in closure of leaflets of the valve. The collar 6 may be used to retain the filaments in the drawn-in configuration by clamping or crimping the collar to the filaments, or using other suitable means.

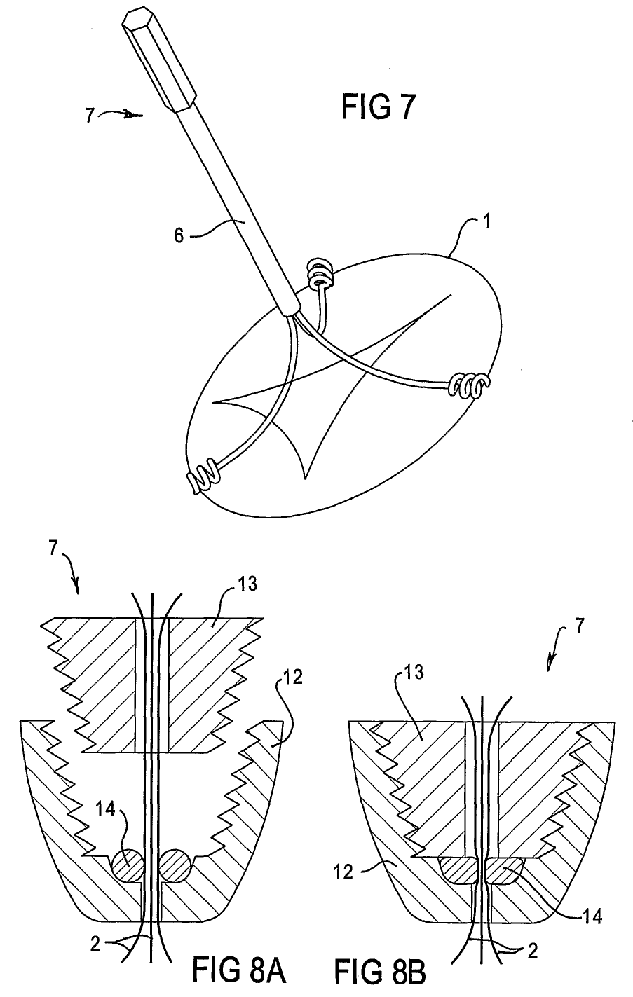

In one embodiment, a locking member is used to retain the filaments in the drawn-in configuration. FIG. 7 illustrates a suitable locking member 7 applied over the filaments at one end of collar 6. Locking member 7 may take any suitable form which can be applied to the filaments and/or move freely along the filaments before being locked in position. Preferably, locking member 7 exhibits no movement relative to the filaments after being locked into position. It may be desirable for the locking member to be releasable to facilitate adjustment of the valve constriction device after implantation.

For percutaneous delivery and deployment of the constriction device, it is desirable that the locking member 7 fits inside catheter 9 or other delivery lumen for percutaneous delivery to the collar 6 and filaments 2. It is to be understood that in certain embodiments, the locking member may be built into the collar 6. One suitable form of locking member 7 may include a set of pie jaws or chuck jaws such as a collet chuck having push or pull back operation, or screw operation enabling the jaws to close in on and clamp, crimp or otherwise lock onto the filaments. Alternatively, the locking member 7 may adopt a ratchet, wedge or clip-type system.

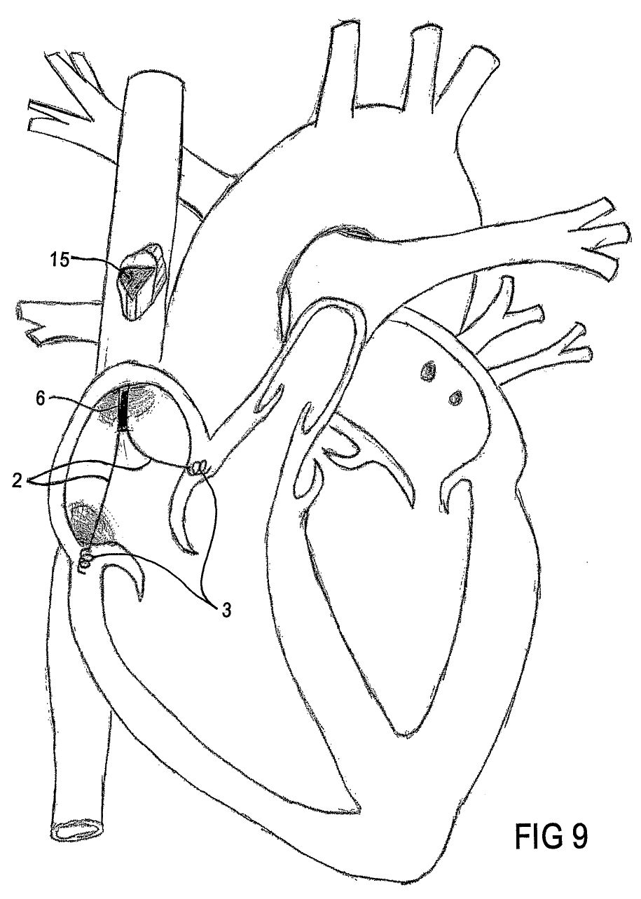

An example is illustrated in FIGS. 8A and 8B. Locking member 7 includes an outer body portion 12 and an inner body portion 13 having complementary screw threads. The inner portion 13 of locking member 7 includes a void for receiving the filaments therein. A washer or seal 14 is provided which, when inner body portion 13 is screwed further into the outer body portion 12, conforms and compresses within the available space inside the locking member, retaining the filaments 2 by way of friction between the filaments 2 and the washer 14. Alternatively, there may be no void in the inner portion 13 in which case the filaments are retained by friction between the filaments and the washer and screw threads of the outer and inner body portions 12,13. As the locking member 7 is in abutment with collar 6, the drawn-in configuration of the engaged tissue is maintained.

In one embodiment, an anchor may be provided to secure one or more of the engaged filaments to a region of sufficiently robust body tissue near the valve to further augment and improve valve function. This is achieved by drawing the filaments and thus the engaged tissue toward the anchor, in addition to (radial) drawing in of the engaged tissue. One example of this arrangement is illustrated in FIG. 9 where filaments 2 which are engaged with the annulus of the tricuspid valve are secured to the lining of a region of the superior vena cava by way of anchor 15. In addition to the drawn-in configuration of the engaged annular tissue created by collar 6, anchoring the filaments in this way has the added advantage of “lifting” the valve annulus relative to the leaflets, which aids sealing of leaflets after treatment with the device.

It is to be understood that the present invention, in its various forms, may achieve an improvement in valve function by augmenting the annulus of the valve being treated by drawing engaged portions of the annulus tissue inward, or by drawing engaged portions of the annulus tissue toward an anchor secured to robust body tissue located near the valve, or using a combination of these approaches.

Whilst implantation of the constriction device will have some effect on the flow characteristics of blood flowing through the valve, patients who suffer from severe valvular insufficiency will still benefit from implantation of the device, despite the potential increase in blood flow turbulence.

Various adaptations may be made to the parts previously described. For example, the collar may be in the form of a locking disc or a locking disc may be used in conjunction with the collar already described. A disc of this kind may be passed over the wires to the valve annulus, and have a hole for each of the filaments. The wires can then be drawn through the disc, thus constricting the valve annulus and the filaments secured to the disc or bound together by a knot or other means. This may be used in addition to an anchoring means for drawing the valve annulus toward the anchor thus further improving valve compliance.

Alternatively or additionally, a tensioning disc may be threaded onto the filaments and tensioned thereon to maintain the drawn-in configuration. Preferably the tensioning disc is a one-way tensioning disc. As another alternative, a physician may deliver a band over the filaments to substantially abut the disc and crimp the band to preclude withdrawal of the filaments through the disc. Use of a band in this way may also be, for example, in conjunction with the collar 6.

To avoid rejection from the body and/or infection or failure of the device, it is preferred that the device is made from a biocompatible material. In one preferred embodiment, the filaments 2 are formed from extrusions of a nickel-titanium alloy such as nitinol. Alternatively, the filaments 2, sheaths 20, support structures 600 and/or other components of the constriction device may be manufactured from other biocompatible metal alloys or materials including stainless steels, ceramics, plastics or other synthetic materials or combinations of these.

Advantageously, patients undergoing valve treatment in accordance with the various embodiments of the present invention need not require general anesthetic. Rather, the patient may be treated by percutaneous access to the valve while sedated. Clearly, this is beneficial to the patient as the recovery time is significantly reduced when compared with existing treatments for valvular insufficiency, and the device may be implanted during an outpatient procedure, reducing costs. Use of a sedative also reduces the risk of mortality which is associated with use of general anesthetic in elderly patients.

Percutaneous treatment of valve failure according to the various embodiments of the present invention eliminates the need for open heart surgery which has previously been required for treating heart valve failure, although the invention may be utilised in an open-heart procedure should the need arise. Advantageously in percutaneous delivery, patients treated according to embodiments of the invention are able to recover more quickly with reduced risk of infection, surgical complications and mortality, and the discomfort which accompanies open heart or other major surgery.

Whilst in most cases it would be desirable to constrict the annulus in such a way that a healthy annulus geometry is restored, in many serious cases of heart valve failure it may be sufficient to achieve an annulus reduction of 25% or less. In many cases, this will restore valve function to a degree which improves the quality of life of the patient. Another advantage of implantation of the inventive device and use of the inventive method is that it minimizes further dilation of the valve annulus. Moreover, the invention in its various embodiments also presents the possibility of adjustment in situ, after initial deployment of and treatment using the device

While some embodiments of the present invention have been illustrated here in detail, it is to be understood that modifications and adaptations to these embodiments may occur to one skilled in the art without departing from the scope of the present invention as set forth in the following claims.

1. A method of treating a disorder associated with a heart valve of a patient, the method comprising the steps of:
engaging tissue at spaced apart locations of an annulus of the valve by attaching at least one of a plurality of filaments to tissue at each of the locations;
drawing the locations radially inward by drawing the filaments inward to a drawn-in configuration; and
securing the filaments in the drawn-in configuration.

2. A method according to claim 1 further including the step of delivering a collar over a length of the filaments, wherein relative movement between the filaments and the collar draws the locations inward.

3-7. (canceled)

8. A method according to claim 1 further including the step of anchoring at least one of the locations to a region of tissue spaced from the annulus so as to draw the at least one of the locations toward the region.

9-12. (canceled)

13. A method according to claim 1 further including the step of using one or more of the filaments to conduct an electric signal to an analysis device for use in positioning the one or more filaments.

14. (canceled)

15. A method according to claim 1 wherein one or more of the filaments includes a marker identifiable from outside the body to facilitate positioning of the filaments prior to engagement with the annular tissue.

16-20. (canceled)

21. A device for treating a disorder associated with a heart valve of a patient, the device including:
a plurality of filaments each having an engaging portion for engaging annular tissue of the valve; and
a collar facilitating a drawn-in configuration of engaged annular tissue by relative movement between the filaments and the collar.

22. A device according to claim 21 wherein each engaging portion includes a helical tip engageable with annular tissue of the valve using a torque tool.

23. A device according to claim 21 wherein each of the filaments further includes a connecting region for releasably connecting a tool configured to facilitate engagement of the engaging portion of each filament with the annular tissue.

24. A device according to claim 21 wherein one or more of the filaments is configured to conduct an electric signal to an analysis device, to facilitate positioning of an associated engaging portion.

25. A device according to claim 21 wherein one or more of the filaments further includes a marker identifiable from outside the patient's body to facilitate positioning of the filaments.

26-27. (canceled)

28. A device according to claim 21 further including a locking member for securing the filaments in the drawn-in configuration.

29. (canceled)

30. A device according to claim 21 further including an anchor to anchor at least one of the engaged filaments to a region of tissue spaced from the valve to facilitate drawing of the engaged tissue toward the region.

31. A device according to claim 21 wherein each of the filaments is tapered toward a smaller cross-sectional area proximal the engaging portion.

32-34. (canceled)

35. Apparatus for delivering a valve annulus constriction device, the apparatus including:
a catheter configured to deliver a plurality of filaments percutaneously to an annulus of a heart valve being constricted and to deliver a collar to be applied over a length of the filaments,
wherein relative movement between the catheter and a filament facilitates positioning of the filament prior to engagement with the valve annulus tissue.

36. Delivery apparatus according to claim 35 wherein the filaments each include an engaging portion, and the catheter is configured to deliver the filaments therein in a staggered arrangement.

37. Delivery apparatus according to claim 35 wherein the relative movement includes retraction of the catheter relative to the filament, thereby causing the filament to splay facilitating engagement with the annulus tissue.

38. Delivery apparatus according to claim 35 further including a percutaneously operable tool configured to facilitate engagement of an engaging portion associated with each of the filaments with the annular tissue.

39-40. (canceled)

41. Delivery apparatus according to claim 35 further including a centering member having a tip locatable on a distal side of the valve being constricted to facilitate centralization of the constriction device relative to the valve annulus.

42. A method of treating a disorder associated with a heart valve of a patient wherein the valve includes an annulus having tissue surrounding a valve opening and valve leaflets at the opening, the method including the steps of:
engaging the tissue at spaced apart engaged locations around the valve opening;
urging the engaged locations to move in a direction substantially perpendicular to a plane of the valve opening to displaced positions; and
anchoring the engaged locations in the displaced positions.

43. A method according to claim 42, further including the steps of drawing the engaged locations radially inward to a drawn-in configuration, and securing the engaged locations in the drawn-in configuration.

44. A device for treating a disorder associated with a heart valve of a patient, the device including:
a plurality of filaments each having an engaging portion for engaging annular tissue of the valve; and
an anchor for anchoring the engaged filaments to a region of tissue spaced from said annulus to facilitate drawing of the engaging portion toward the anchor.

45. A device according to claim 44 further including a collar facilitating a radially inward movement of said engaging portions by relative movement between the filaments and the collar.

46. A method according to claim 43 wherein the engaged locations are engaged by attaching a filament to tissue at each of the locations and drawing the filaments in the direction substantially perpendicular to a plane of the valve opening.

47. A method according to claim 43 wherein the engaged locations are engaged by attaching a filament to tissue at each of the locations and drawing the filaments inwardly within the valve opening.
